(19) United States
(12) Patent Application Publication
(10) Pub. No.: US 2013/0079865 A1
(43) Pub. Date: Mar. 28, 2013

(54) IMPLANT AND METHOD FOR MANUFACTURING THE SAME

(71) Applicants: Stefanie Poehlmann, Rostock (DE); Alexandre Amido, Rostock (DE)

(72) Inventors: Stefanie Poehlmann, Rostock (DE); Alexandre Amido, Rostock (DE)

(73) Assignee: BIOTRONIK AG, Buelach (CH)

(21) Appl. No.: 13/614,427

(22) Filed: Sep. 13, 2012

Related U.S. Application Data
(60) Provisional application No. 61/538,152, filed on Sep. 23, 2011.

(51) Int. Cl. A61F 2/82

(52) U.S. Cl. 623/1.15

(57) ABSTRACT

An implant comprising a perforated, preferably hollow-cylindrical basic structure which has at least one section, wherein in each section at least one strut is arranged. In order to achieve a more homogenous coverage of the wall of the treated vessel, a good interlock with the wall of the treated body cavity and a better distribution of the mechanical stresses and elongations, the strut has a multiplicity of identical, simple and interconnected elements, the basic shape of which is substantially similar to the shape of base line elements of an imaginary first order base line which likewise runs in the section, wherein the imaginary first order base line represents a reference line for the arrangement of the elements and the elements are not more than half the size of the base line elements.

### CROSS REFERENCE TO RELATED APPLICATIONS

This invention claims priority to U.S. provisional patent application Ser. No. 61/538,152 filed Sep. 23, 2011. The contents of which are herein incorporated by reference its entirety.

### TECHNICAL FIELD

The present invention relates to an implant, in particular and intraluminal endoprosthesis having a perforated, preferably hollow-cylindrical basic structure which has at least one section, wherein in each section at least one strut is arranged.

### BACKGROUND

Medical endoprostheses or implants for many different applications are known in a large variety from the prior art. Implants in the meaning of the present invention are to be understood as endovascular prostheses or other endoprostheses, for example stents (vascular stents (vascular stent including cardiac stent and cardiac valve stent, e.g. mitral stent, pulmonary valve stent), bile duct stents), endoprostheses for closing patent foramen ovales (PFO), stent grafts for treating aneurysms, endoprostheses for closing an ASD (atrial septal defect) as well as prostheses in the region of the hard and soft tissue.

Today, stents serving for treating stenoses (vascoconstrictions) are most frequently used as implants. They have a perforated tubular or hollow-cylindrical basic structure which is open at both longitudinal ends. The basic structure of conventional stents is often composed of individual meshes which are formed from differently shaped struts, for example zigzag struts or meander-shaped struts. Such an implant is often inserted into the vessel to be treated by means of a catheter and serves for supporting the vessel over a prolonged time period (months or years). By the use of stents, constricted regions in the vessels can be dilated resulting in a lumen gain.

For releasing drugs from such implants (for example in case of a so-called drug eluting stent (DES)) into the wall of a treated vessel it is intended that the implant achieves coverage of the vessel wall as homogenous as possible. Furthermore, migrating of the stent in vivo (e.g. in the artery) is to be prevented and the strength of the stent under the load of the corresponding vessel is to be increased. Moreover, the radiopacity of the implants could be improved in order to be able to monitor the placement of the implants and their behavior in vivo from time to time and in a simple manner; currently available thin struts of stents very often have only a poor radiopacity.

### SUMMARY

The object of the present invention therefore is to provide an implant which has the above-mentioned improvements.

The above object is achieved by an implant with the features of the claim 1.

The at least one strut of a section of an implant according to the invention has in particular a multiplicity of identical, simple and interconnected elements, the basic shape of which is substantially similar to the shape of base line elements of at least one imaginary first order base line which likewise runs within the section, wherein the at least one imaginary first order base line represents a reference line for the arrangement of the elements and the elements are not more than half the size of the base line elements. The last mentioned feature means that in at least one dimension, preferably in the dimension along the base line element, the elements have a dimension which is not more than half the size of the one of the base line elements. This means that in the case that the dimension of the element along the base line element is designated as length of the element, the elements have only half the length of the base line elements. The height of the elements can vary and is preferably also half the height of the base line elements.

In other words, the at least one strut of the implant, which strut is arranged in a section, is formed from a plurality of elements. These elements have the same basic shape which is substantially similar to the one of which an imaginary base line is composed. The base line element serves in the respective section for forming a reference line for the arrangement of the elements of the at least one strut. Since the basic shape of the strut elements and the shape of the base line elements arranged along the base line are substantially similar to each other, the structure according to the invention of the implant virtually generates a fractal (self-similar) structure. The (small) elements form a strut which shows a delicate structure.

With the reference to the term “basic shape” in connection with the elements of the struts it to is intended to illustrate, besides the use of simple shapes, that the strut width or strut thickness is disregarded for the consideration of the shape of the strut element and for the comparison with the shape of the base line element. According to this, for example, the shape of a center line through the element, or of an upper or outer boundary line, or a lower and inner boundary line along the element is considered as basic shape. In other words, e.g. in case of a rhombus-shaped element of a strut, only the (mathematically two-dimensional) shape of the rhombus with respect to the corresponding base line element is considered and the width and thickness of the sides of the rhombus are neglected. Preferred basic shapes are the wave arch which is preferably formed as sine arch, the rhombus and the zigzag element.

Here, “substantially similar” is to be understood not only as the strict mathematical-geometrical similarity but also such cases are included in which the shape of the strut elements and the shape of the base line elements have the same basic shape (wave, triangle, rhombus, sine, etc.) but deviate in individual dimensions, curvature radii or angles from each other compared to the strict mathematical-geometrical similarity. For example, the base line elements as well as the strut elements can be configured as rhombuses, wherein the base line rhombus angles which correspond to each other and the strut element rhombuses are not identical as they are in case of the strict mathematical-geometrical similarity, but are configured in a slightly different manner.

Preferred curvature radii for a wave arch-shaped element of a strut lie between 0.035 mm and 1 mm. The length of an element of a strut (along the respective basic element) is preferably between 0.1 mm and 5 mm. The angle between two sides of a rhombus element or a zigzag element can lie in the range between 0.5° and 175°.

The configuration according to the invention of the implant has decisive influence on its mechanical properties. First of all, the configuration can be varied in various ways and thus, different advantageous properties of the implant can be combined. Due to the composition of the struts from the individual elements, a higher and more homogenous coverage of the wall of the treated body cavity can be achieved. This is in particular advantageous for the use as implant with drug administration. Furthermore, a good “interlock” with the wall of the treated body cavity can be achieved so that a migration of the implant in the body cavity is prevented. Due to the fine structure of the at least one strut, the latter allows movement in different directions so that a higher flexibility of the implant can be achieved. Mechanical stresses and elongations which act on the implant in the treated body cavity are better distributed than in conventional designs. The lever ratios are changed which results in an increase of the strength with respect to the conventional implant structure. Moreover, a dilatation reserve is provided so that an additional over-dilatation is possible. A cost advantage in particular for braided stents results from the fact that the at least one strut is formed in each case from the same elements.

In one exemplary embodiment, the at least one section of the implant has an annular or helical shape. When using annular sections it is necessary in most cases to interconnect adjacent sections by means of connecting struts. In case of a helically shaped configuration it can be necessary to interconnect the struts of the helix regions arranged on top of each other by means of connecting struts.

Accordingly, in a further exemplary embodiment, the at least one strut of a section or a region of said section is connected to the at least one strut of the adjacent section or an adjacent region of the section by means of at least one connecting strut which is preferably S-shaped. Hereby, a better distribution of the forces and a higher stability is achieved. Preferably, the connecting struts are connected to the respective strut where the curvature of the respective strut has a local extremum (minimum or maximum). Alternatively, a connecting strut can be arranged along the strut in the region of the middle (with respect to the length of the strut) between two adjacent connecting regions to adjacent connecting struts. Such connecting struts are designated as middle-to-middle connector.

Particularly suitable, simple and easily manufacturable elements or base line elements for an implant according to the invention have the shape of a wave shape arch, preferably of a sine arch, a rhombus, a triangle, a zigzag element or the like.

The imaginary first order base line forms a reference line for the arrangement for the elements of the at least one strut in such a manner that the elements in the section are arranged along the at least one imaginary first order base line.

Alternatively, the elements are arranged along at least one imaginary second order base line, wherein the imaginary second order base line is provided in such a manner that it comprises those base line elements which are arranged with a size of not more than 50% is along the at least one first order base line. In this case it is of advantage if the elements of the at least one strut have only a size of not more than 25% of the size of the base line elements which form the at least one imaginary first order base line (at least for the dimension along the base line; the dimension perpendicular to the base line can vary).

As a further alternative, the elements of the at least one strut are arranged along an imaginary third order base line, wherein initially at least one second order base line is provided which comprises those base line elements which are arranged with a size of not more than 50% along the first order base line, and wherein further at least one third order base line is provided which comprises base line elements which are arranged with a size of not more than 25% along the second order base line. In this case, the elements of the at least one strut have a size of not more than 12.5% of the size of the base line elements of the first order base line (all measurements at least for the dimension along the base line; the dimension perpendicular to the base line can vary). In particular the last-mentioned variant of the arrangement of the elements results in a very fine implant structure through which a very homogenous coverage of the wall of the respective treated body cavity is achieved.

The series of the above-described exemplary embodiments of implants having at least one imaginary first order base line, at least one imaginary second order base line, at least one third order base line can be continued correspondingly in an analog manner with at least one imaginary fourth order base line. With each imaginary base line of a higher order, the structure of the implant struts becomes more delicate.

A further variability with respect to the configuration of the implant can be achieved in that the elements or base line elements are arranged in such a manner along the at least one first order base line, or the at least one second order base line, or the at least one third order base line, etc. that the respective base line represents a central line or an upper or left boundary line or a lower or right boundary line.

The implant according to the invention is explained hereinafter in exemplary embodiments by means of figures. All described and/or figuratively illustrated features form the subject matter of the invention, independently of their combination in the claims or their relations to each other.

### DESCRIPTION OF THE DRAWINGS

In the figures, schematically:

FIG. 1 shows three sections of a first exemplary embodiment of an implant according to the invention in a perspective side view,

FIG. 2 shows a cut-out of a flat projection of a second exemplary embodiment of an implant according to the invention in a side view,

FIG. 3 shows a cut-out of the imaginary first order base line of the flat projection (FIG. 3a) and a region of a strut (FIG. 3b) of the second exemplary embodiment illustrated in FIG. 2 in a top view,

FIG. 4 shows a cut-out of the imaginary first order base lines (FIG. 4a) and a region of a strut (FIG. 4b) of a flat projection of a third exemplary embodiment of an implant according to the invention in a top view,

FIG. 5 shows a cut-out a flat projection of a fourth exemplary embodiment of an implant according to the invention in a top view,

FIG. 6 shows a cut-out of a flat projection of a fifth exemplary embodiment of an implant according to the invention in a top view,

FIG. 7 shows an enlarged cut-out of the flat projection illustrated in FIG. 6,

FIG. 8 shows a region of a strut (FIG. 8a) and a cut-out of the flat projection (FIG. 8b) of a sixth exemplary embodiment of an implant according to the invention in a top view, and

FIG. 9 shows a region of a strut (FIG. 9a) and a cut-out of the flat projection (FIG. 9b) of a seventh exemplary embodiment of an implant according to the invention in a top view.

### DETAILED DESCRIPTION

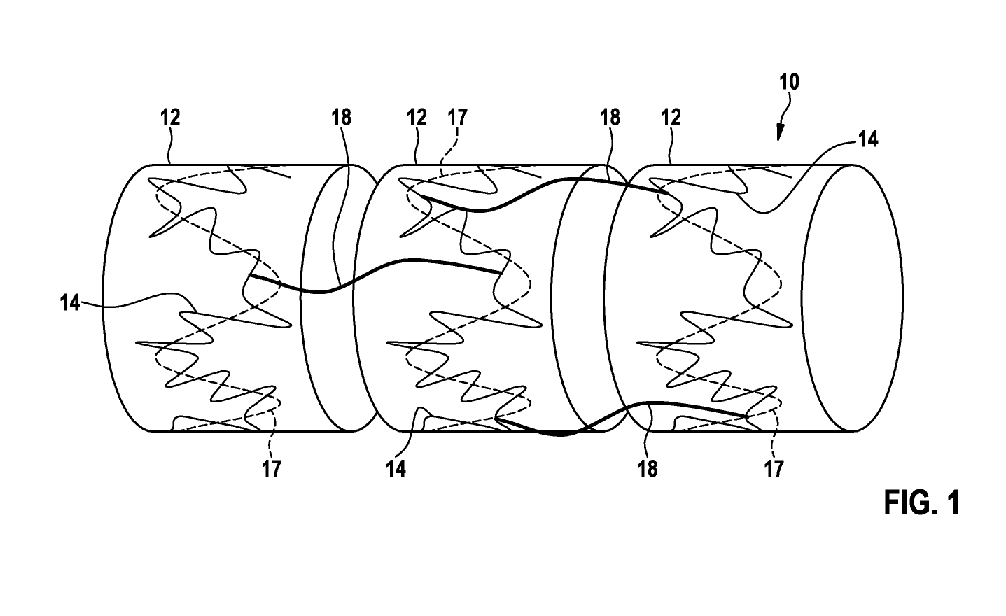

FIG. 1 shows three sections 12 of a first exemplary embodiment of an implant according to the invention which is configured as a stent 10. Each annular section 12 has in each case one strut 14 which extends in a wave-shaped, meander-like manner along an imaginary wave-shaped base line 17 (dashed line). Preferably, the base line 17 has a sinusoidal shape. The base line 17 as well as the strut 14 extends in a wave-shaped manner such that they have alternating mountains and valleys or have in each case curvature radii which are proximally and distally oriented in an alternating manner.

It is to be explicitly noted that the annular sections indicated by the reference number 12 in FIG. 1 are merely imaginary sections which serve for illustrating the struts 14.

The strut 14 is composed of wave arch-shaped elements which are arranged along the imaginary base line 17 in such a manner that the base line 17 forms an imaginary center line. The base line 17 is also composed of individual wave arches, preferably sine arches, as base line elements. Each wave arch of the strut 14 is connected to the next wave arch of the strut 14. Also, the wave arch-shaped base line elements of the base line 17 are connected to each other.

The length of a wave-shaped or S-shaped strut elements is approximately one sixth of the length of a wave-shaped or S-shaped base line element. In case of the base line element, the length corresponds to the extension in a direction perpendicular to the longitudinal direction of the stent and in case of the strut element, the length corresponds to the extension along the base line element.

In the region of the respective extrema (minimum, maximum) in the curvature along the imaginary base line 17, connecting struts 18 are arranged each of which connect the strut of a first section 12 to the strut 14 of an adjacent section 12. The connecting struts 18 which extend substantially in the longitudinal direction of the stent serve for absorbing forces acting on the strut in the longitudinal direction (direction of the longitudinal axis). Each connecting strut 18 preferably forms a wave arch which in each case has a mountain and a valley or, respectively, the curvature radii extend alternatingly in opposite directions perpendicular to the longitudinal direction of the stent. Different types of connecting struts 18 can be used on the same stent, e.g. two different types of connecting struts 18 with different curvature radii, e.g. a first type of connecting struts 18 between the first section 12 with a strut 14 and the second section 12 with a strut 14, a second type of connecting struts 18 between the second section 12 and a third section 12 with a strut 14, and a first type of connecting struts 18 between the third section 12 and a fourth section 12 with a strut 14, etc.

For this and all exemplary embodiments described below, the stent can be manufactured as so-called slotted tube stent. This means that the cavities between the individual struts are cut out of the raw material, a tube, by means of a laser. Alternatively, the stent can be manufactured as so-called braided stent. In this case, the stent is braided from wire. However, other manufacturing methods are also conceivable, e.g. welding together individual parts of the stent or rapid prototyping.

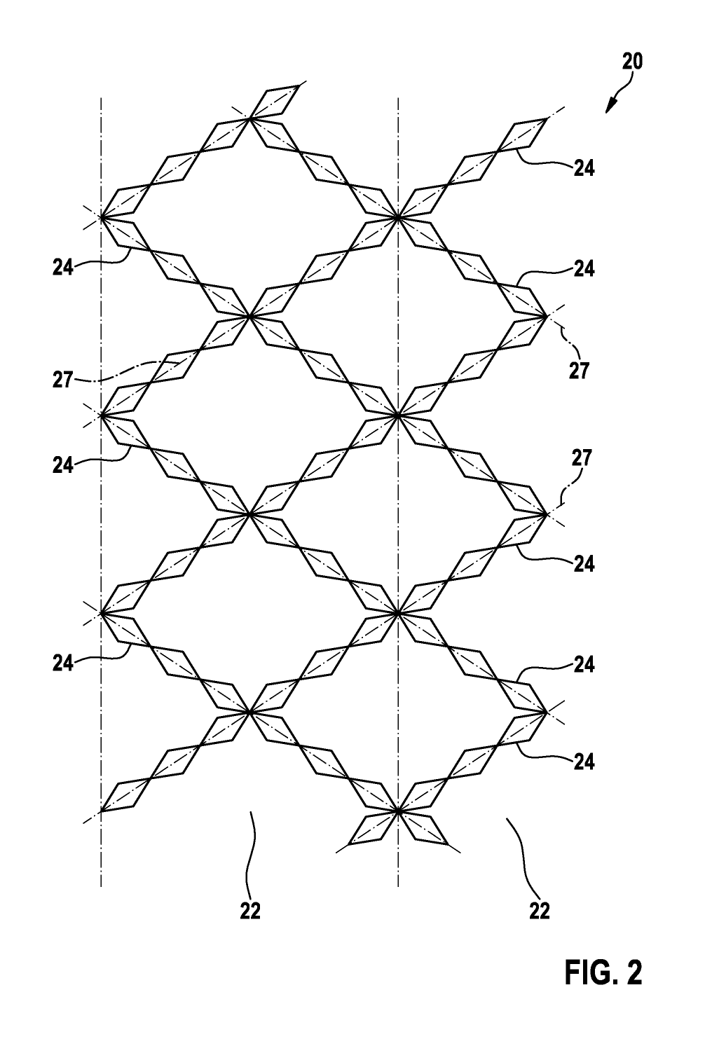

FIG. 2 illustrates the flat projection of a second exemplary embodiment of a stent. Each annular section 22 has struts 24 which form rhombus-shaped meshes along imaginary base lines 27 (dashed lines). In the illustration, the annular sections 22 are delimited from each other in each case by a dotdashed line.

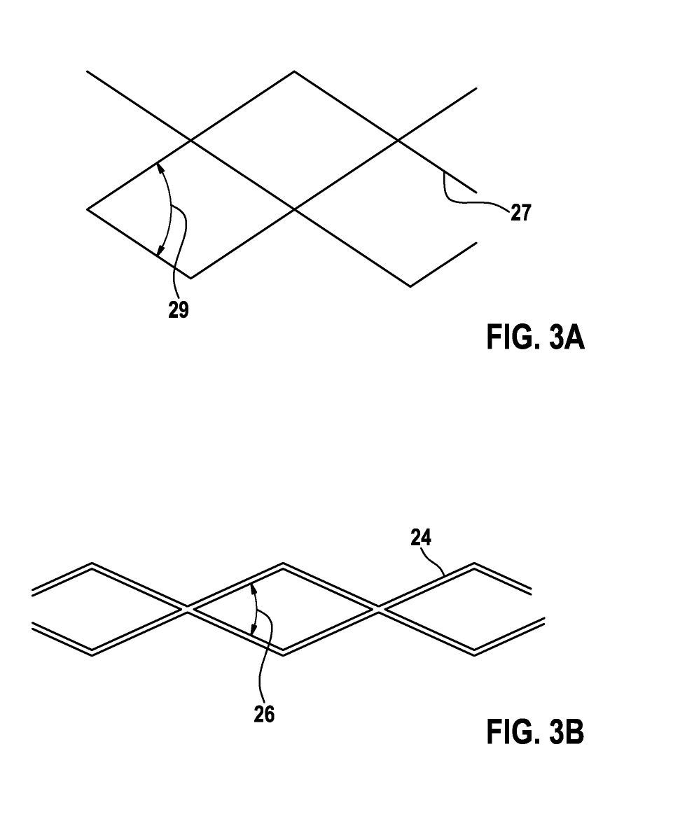

On each side of the rhombuses from the imaginary base lines 27, the struts 24 extend which consist of small rhombuses arranged in series. Each small rhombus forms a strut element. The shape of the base line elements (rhombus meshes) and the elements of the struts 24 are shown again individually in FIG. 3, wherein FIG. 3a illustrates the arrangement of the imaginary first order base lines 27 while FIG. 3b shows the struts 24 in an enlarged illustration. The imaginary base lines 27 form a center line through the rhombus elements of the struts 24.

By means of FIG. 3 it is clearly shown that the second exemplary embodiment represents a variant in which the base line elements (rhombuses), within the strict mathematical-geometrical meaning, are not similar to the rhombuses of the struts 24. The reason for this is that the acute inner angle 26 of the rhombuses of the struts 24 is smaller than the acute inner angle 29 of the rhombuses which form the imaginary base lines 27. The second exemplary embodiment is therefore designated as fractal-like structure.

The length of the rhombuses of the struts 24 is approximately one sixth of the length of the rhombuses which form the base line. In case of the base line element, the length corresponds to the extension in the longitudinal direction of the stent and in case of the strut element, the length corresponds to the extension along the respective side of the base line element.

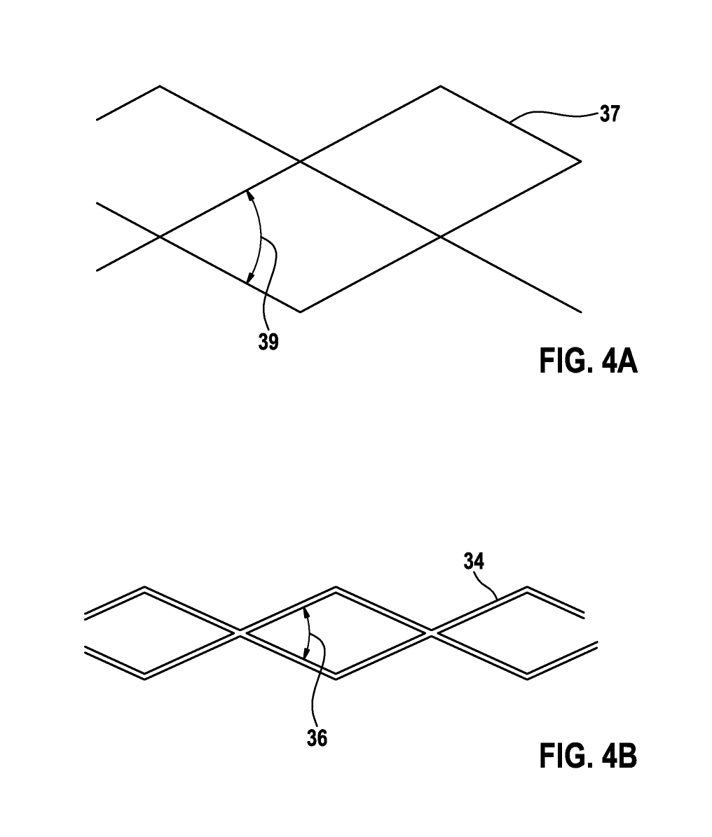

In contrast to that, FIG. 4 illustrates a third exemplary embodiment in which, within the mathematical meaning, the rhombuses of the struts 34 are similar to the rhombuses of which the imaginary base lines 37 are composed. The similarity of the rhombuses is determined in that the acute inner angle 36 of the strut elements is equal to the acute inner angle 39 of the rhombuses of the base line 37. An implant with the struts 34 which, analogous to FIG. 2, are arranged along the imaginary base lines 37 thus forms a fractal structure.

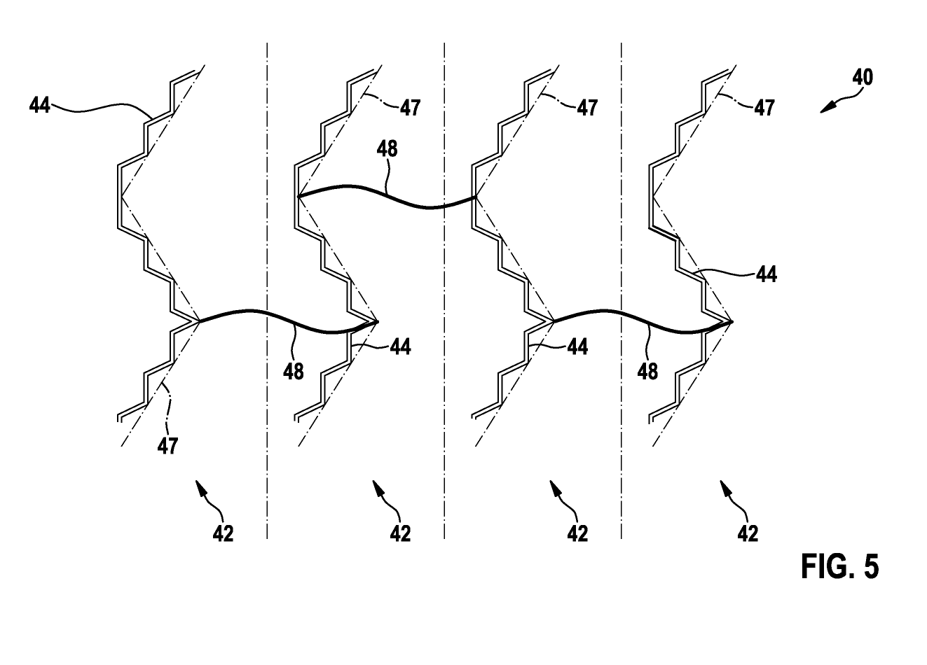

FIG. 5 shows a fourth exemplary embodiment in which in each annular section 42 of the stent 40, struts 44 are arranged which are composed of zigzag elements arranged side by side, wherein each element contains two adjacent sides of a triangle. The zigzag elements of the respective strut 44 are arranged along an imaginary base line 47 drawn with a dashed line which extends in the entire section 42 on the right side of the zigzag elements of the strut 44. Each imaginary base line 47 also consists of zigzag elements arranged side by side. Analogous to the first exemplary embodiment, S-shaped connecting struts 48 are provided in the region of the respective minima or maxima of the struts 44, each of which connecting struts connects in each case a strut 44 of a first section 42 to a strut 44 of an adjacent section 42 and extends substantially in the longitudinal direction.

The length of a zigzag-shaped strut element is approximately one sixth of the length of a zigzag-shaped base line element. In case of the base line element, the length corresponds to the extension in a direction perpendicular to the longitudinal direction of the stent and in case of the strut element, to the extension along the base line element.

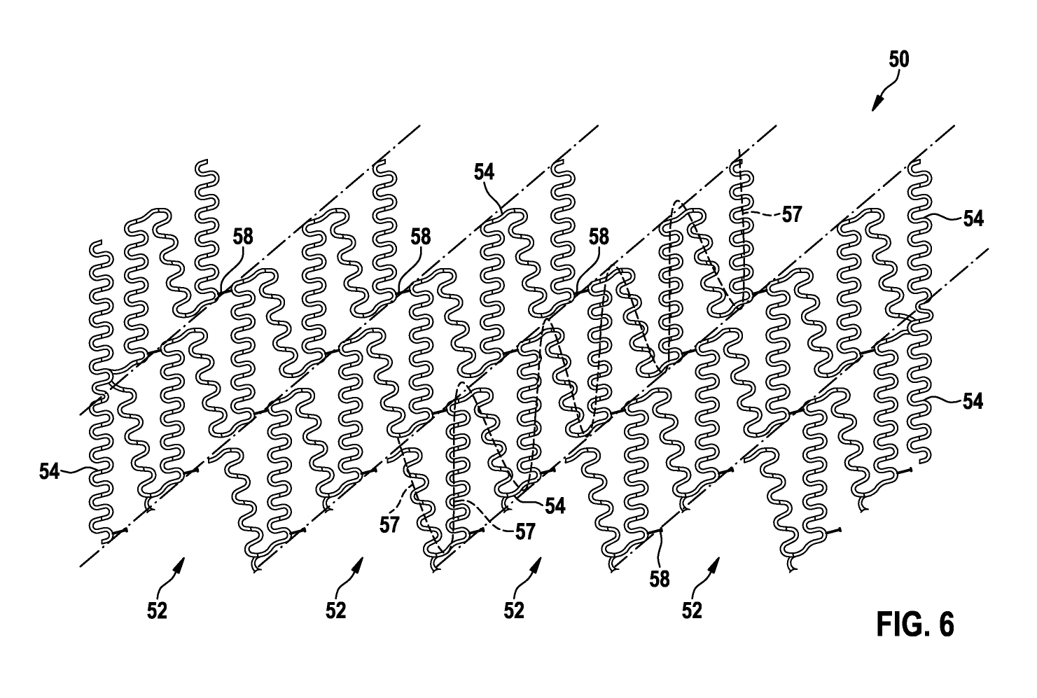

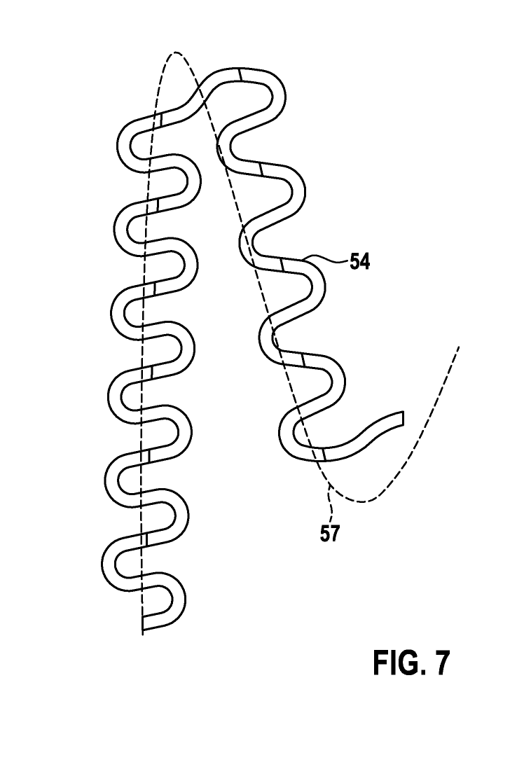

The fifth exemplary embodiment illustrated in FIGS. 6 and 7 has struts 54 which have wave arch-shaped elements arranged side by side along an imaginary base line 57. The imaginary (dashed) base line is also composed of wave arch-shaped base line elements which are arranged side by side. The cut-out illustrated in FIG. 7 shows particularly clear that the elements of the strut 54 are only substantially similar to the shape of the elements of the base line 57. The base line 57 forms substantially a center line for the arrangement of the elements of the strut 54. In this embodiment too, the struts 54 of adjacent helically shaped sections 52 are connected to each other via short connecting struts 58 substantially extending in the longitudinal direction.

The length of a wave arch-shaped strut element of the exemplary embodiment illustrated by means of the FIGS. 6 and 7 is approximately one twelfth of the length of a wave arch-shaped base line element. In case of the base line element, the length corresponds to the extension in a direction oblique to the longitudinal direction of the stent (see dotdashed lines) and in case of the strut element, the length corresponds to the extension approximately along the base line element.

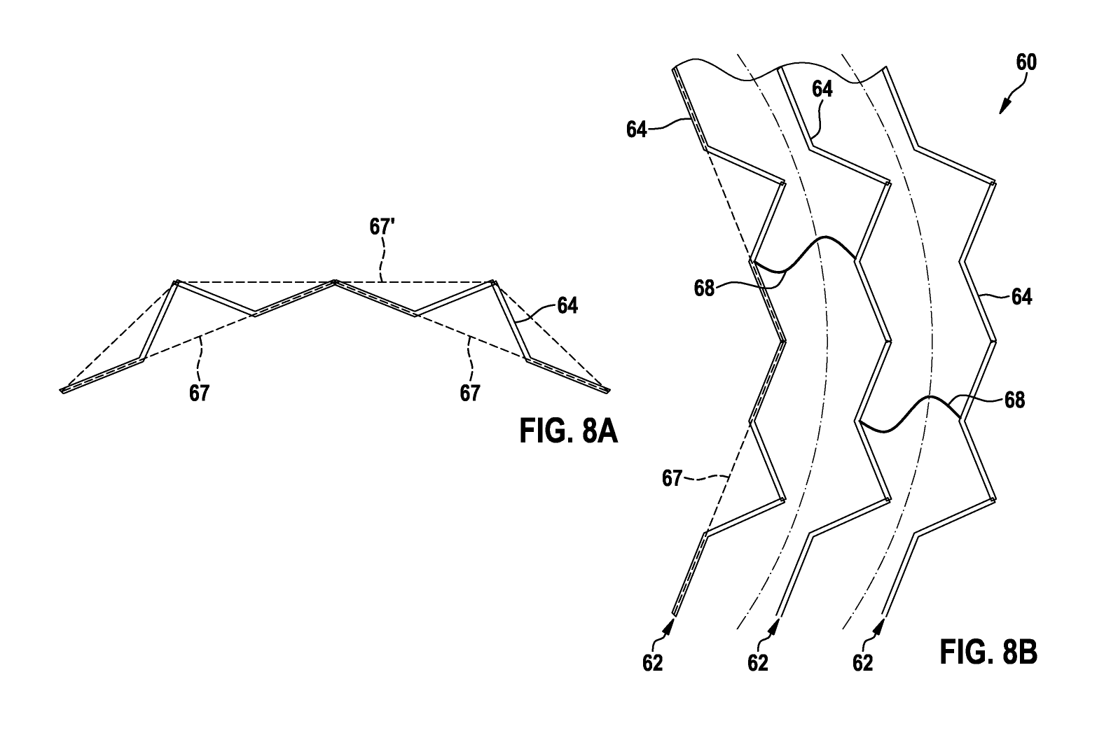

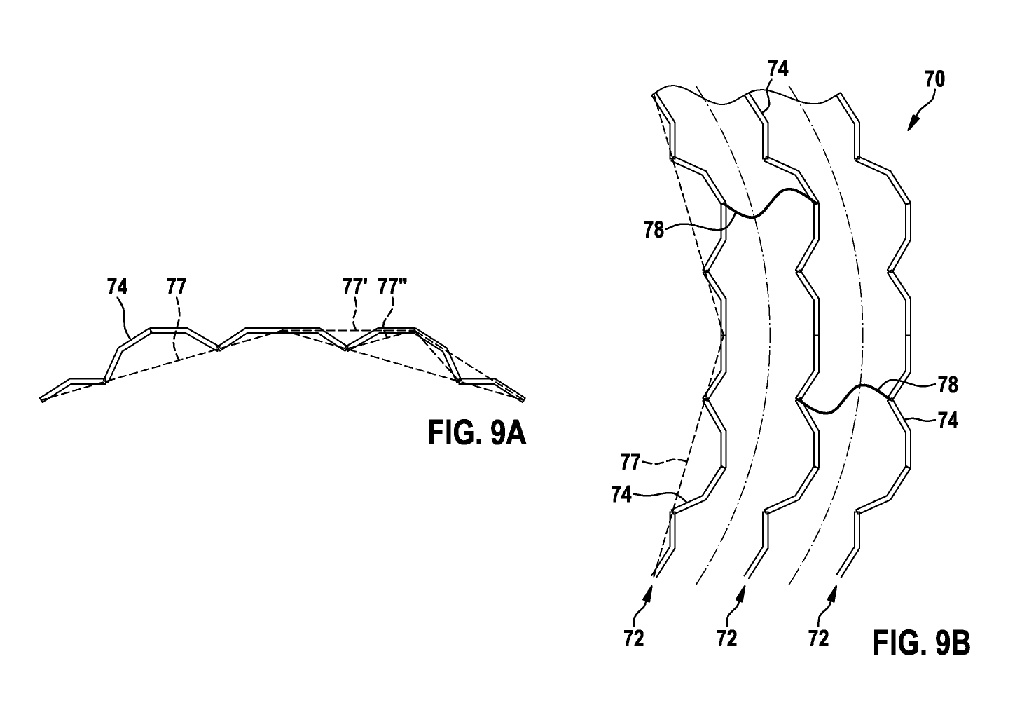

The sixth and seventh exemplary embodiments shown in FIGS. 8 and 9 contain further fractal planes.

In the sixth exemplary embodiment shown in FIG. 8, an imaginary second order base line 67′ was formed which is based on a base line 67 formed from zigzag elements adjoined in series and which is likewise composed of said zigzag elements which, however, have only 50% of the size of the zigzag elements of the first order base line 67. Along this imaginary is second order baseline 67′, the likewise zigzag-shaped elements of the strut 64 are now being arranged. They have a size of only approximately 25% compared to the zigzag elements of the first order base line 67. The zigzag elements of the first order baseline 67 are similar to the zigzag elements of the imaginary second order base line 67′ and to the zigzag elements of the strut 64.

The length of a zigzag-shaped strut element is approximately one fourth of the length of a zigzag-shaped element of the first order base line 67. In case of the base line element of the first order base line 67, the length corresponds to the extension in a direction perpendicular to the longitudinal direction of the stent and in case of the strut element, the length corresponds to the extension along the base line element of the second order base line 67′. The length of a zigzag-shaped element of the second order base line 67′ is approximately half the length of a zigzag-shaped element of the first order base line 67. In case of the element of the second order base line 67′, the length corresponds to the extension along the respective element of the first order base line 67.

In the exemplary embodiment of an implant according to the invention illustrated in FIG. 9, another fractal plane is added. From an imaginary first order base line 77, an imaginary second order base line 77′ is formed analogously to the procedure of the exemplary embodiment shown in FIG. 8. Analogously, an imaginary third order base line 77″ is formed from the second order base line 77′, wherein the elements of the third order base line 77″ are similar to the zigzag elements of the first order base line 77 and the elements of the strut 74. The imaginary third order base line 77″ serves as a basis line for the arrangement (side by side) of the zigzag-shaped elements of the strut 74. The zigzag elements of the struts 74 have a size of approximately 12.5% compared to the size of the elements of the imaginary first order 77. Each strut 74 is arranged in an annular section 72 of the implant 70.

The length of a zigzag-shaped element of the strut 74 is approximately one eighth of the length of a zigzag-shaped element of the first order base line 77. In case of the base line element of the first order base line 77, the length corresponds to the extension in a direction perpendicular to the longitudinal direction of the stent and in case of the strut element, the is length corresponds to the extension along the base line element of the third order base line 77″. The length of a zigzag-shaped element of the second order base line 77′ is approximately half the length of a zigzag-shaped element of the first order base line 77 and the length of a zigzag-shaped element of the third order base line 77″ is approximately one fourth of the length of a zigzag-shaped element of the first order base line 77. In case of the element of the second order base line 77′, the length corresponds to the extension along the respective element of the first order base line 77 and in case of the element of the third order base line 77″, the length corresponds to the extension along the respective element of the second order base line 77′.

The struts 74 of adjacent sections 72 are connected to each other in the longitudinal direction by means of S-shaped connecting struts 78. Analogously, the sixth exemplary embodiment shown in FIG. 8 also has connecting struts 68 which connect the struts 64 of adjacent sections 62 to each other in the longitudinal direction.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

Reference list

| | |
|---|---|
| 10, 20, 30, 40, 50, 60, 70 | Stent |
| 12, 22, 32, 42, 52, 62, 72 | Section |
| 14, 24, 34, 44, 54, 64, 74 | Strut |
| 17, 27, 37, 47, 57, 67, 77 | Imaginary first order base line |
| 67′, 77′ | Imaginary second order base line |
| 77″ | Imaginary third order base line |
| 18, 48, 58, 68, 78 | Connecting strut |

What is claimed is:

1. An implant comprising a perforated, hollow-cylindrical base structure which has at least one section, wherein in each section at least one strut is arranged, characterized in that the strut has a multiplicity of identical, simple and interconnected elements, a basic shape of which is substantially similar to a basic shape of base line elements of at least one imaginary first order base line which also runs in the section, wherein the at least one imaginary first order base line provides a reference line for the arrangement of the elements and the elements are not more than half the size of the base line elements.

2. The implant according to claim 1, characterized in that the at least one section is annularly shaped or helically shaped.

3. The implant according to claim 1, characterized in that the at least one strut of a section or a region of the section is connected to the at least one strut of the adjacent section or an adjacent region of the section by means of at least one connecting strut which is optionally S-shaped.

4. The implant according to claim 1, characterized in that the basic shape of at least one of the elements and at least one base line element is selected from the group consisting of a wave arch, a rhombus, a triangle, and a zigzag.

5. The implant according to claim 1, characterized in that the elements are arranged along the at least one imaginary first order base line.

6. The implant according to claim 1, characterized in that at least one imaginary second order base line is provided which comprises the base line elements which are arranged with a size of not more than 50% along the first order base line and the elements are arranged along an imaginary second order base line.

7. The implant according to claim 1, characterized in that at least one imaginary second order base line is provided which comprises the base line elements which are arranged with a size of not more than 50% along the first order base line, and that further at least one third order base line is provided which comprises base line elements which are arranged with a size of not more than 25% along the second order base line and the elements are arranged along the at least one third order base line.

8. The implant according to claim 5, characterized in that the elements or base line elements are arranged in such a manner along the at least one first order base line or the at least one second order base line or the at least third order base line that the respective base line represents a central line or an upper boundary line or a lower boundary line.
